(19) United States
(12) Patent Application Publication
(10) Pub. No.: US 2007/0248678 A1
(43) Pub. Date: Oct. 25, 2007

(54) Uniform-sized hydrophilic metal oxide nanoparticles and preparation method thereof

(75) Inventors: Kyoungja Woo, Seoul (KR); Jong-Ku Park, Seoul (KR); Jae-Pyoung Ahn, Seoul (KR); Jangwon Hong, Seoul (KR)

Correspondence Address: DARBY & DARBY P.C., P.O. BOX 770, Church Street Station, New York, NY, 10008-0770, US

(73) Assignee: Korea Institute of Science and Technology, Seoul (KR)

(21) Appl. No.: 11/235,915

(22) Filed: Sep. 26, 2005

(30) Foreign Application Priority Data
Oct. 1, 2004 (KR) 10-2004-0078321

(51) Int. Cl. A61K 9/14; B32B 15/02

(52) U.S. Cl. 424/489; 977/906; 428/548

(57) ABSTRACT

The present invention provides for a metal oxide nanoparticle that contains a metal core, a shell formed on the surface of the core and consisted of the same metal as the core, and an organic compound containing an element capable of covalently bonding with the nanoparticle and a hydrophilic functional group. According to the examples, uniform-sized hydrophilic metallic oxide-based nanoparticles are obtained when superparamagnetic iron oxide particles, which have a globular shape and are less than 20 nanometers in size, are first synthesized in an organic solution, and then are converted to hydrophilic particles after undergoing surface modification.

### BACKGROUND OF THE INVENTION

1. Field of the Invention

The present invention relates to hydrophilic metal oxide nanoparticles that are uniform in size, and particularly, to surface-modified hydrophilic metallic oxide-based nanoparticles that have an improved dispersity and the method of preparation thereof.

According to the present invention, dispersity of many types of hydrophobic metallic oxide nanoparticles in an aqueous solution can be increased, thereby allowing the nanoparticles to be applied in a wide range of areas including clinical applications.

2. Description of the Background Art

When metal oxide particles are synthesized in an organic solution containing surfactants, the surfactants protect the surface of the particles from agglomerating. However, when the particles are placed in a polar solvent, the protecting layer on the surface of the particles is stripped off and the particles begin agglomerating and forming precipitates. In order to acquire chemical stability and dispersity of the particles in an aqueous solution, there should be a chemical covalent bonding, rather than a physical interaction, between an organic ligand containing a hydrophilic functional group and the particle. Nonetheless, the particles do not make a covalent bond with an organic ligand because the surface of a metal oxide is not reactive.

Iron oxide nanoparticles, such as γ-Fe₂O₃ (maghemite) and Fe₃O₄ (magnetite), have been broadly studied because of their practical applications in the areas such as magnetic resonance imaging (MRI), cell separation and purification, and drug delivery due to their magnetic property and chemical stability. To be used in a number of clinical applications, iron oxide nanoparticles must satisfy the following requirements: having a size less than 20 nanometers and a spherical shape, being uniformly distributed in size, being superparamagnetic, not having any toxicity, having dispersity in an aqueous solution, being biocompatible, having desired targeting, and more. According to a recent study, it is not difficult to obtain spherical superparamagnetic particles less than 20 nanometers in size. Biocompatibility and target functionality are the requirements that are to be acquired after achieving the rest of the above-mentioned requirements.

Among the above-mentioned requirements, a nanoparticle that can be uniformly distributed thus is easily controllable in vivo application, and that is dispersible in an aqueous solution such as a body fluid has never been reported. Conventionally, even though preparation in an aqueous solution allows the particles to obtain a hydrophilic property, the particulate uniformity is reduced. On the other hand, a recently-developed preparation technique in an organic solution can make the particles to be uniformly distributed, but they still face the problem of particle agglomeration and precipitation in an aqueous solution due to their hydrophobic surface property.

Therefore, it would be desired that particulate uniformity is firstly obtained in an organic solution, and then a surface modification is followed in order to convert hydrophobic nanoparticles into hydrophilic nanoparticles prior to proceeding next steps. As an effort to achieve such an effect, a recent study showed clinical applications that use hydrophobic nanoparticles that are physically coated by a hydrophilic and biocompatible polymer. However, the resulting nanoparticles of such technique still couldn't overcome the problem of particle agglomeration and precipitation due to chemical instability caused by a weak linkage by electrostatic interaction, coordinative interaction or van der Waals forces.

Another method is coating an iron oxide nanoparticle with hydrophilic silica prior to bonding its surface with a compound, such as 1-aminopropyl trimethoxysilane, which exposes its amino group, thereby allowing the particulate hydrophilic property. However, this method is also left with the problem of a number of particles being coated together rather than individually.

### SUMMARY OF THE INVENTION

Therefore, an object of the present invention is to provide metal oxide nanoparticles with an increased uniformity of size and dispersity in water.

Another object of the present invention is to provide metal oxide nanoparticles, which have a spherical shape and are less than 20 nanometers in size, uniformly sized, superparamagnetic, chemically stable, well dispersed in an aqueous solution and more, provide an essential basic structure that can detect and treat diseases with a higher sensitivity.

To achieve such an object, the present invention provides metal oxide nanoparticles that comprises a metal oxide core, coated by a shell consisted of the same metal element as the core; and an organic compound that contains a hydrophilic functional group and a covalently bonding thiol group that bonds with the metal element of the shell.

In addition, the present invention provides a method that comprises preparing metal oxide nanoparticles by making the organometallic precursors undergo thermal decomposition and oxidation processes in an organic solution containing surfactants; obtaining metal-rich layer on the surface of the nanoparticles by adding more precursors to a solution containing the nanoparticles under inert condition and making the mixture undergo thermal decomposition; and establishing covalent bonds between the metal element of the nanoparticles and sulfur element of the organic compounds by adding the organic compounds to the solution containing the nanoparticles.

### BRIEF DESCRIPTION OF THE DRAWINGS

FIG. 1 is a diagram of the structure of metal oxide nanoparticles according to the present invention.

FIG. 2 is a diagram that shows the process of making metal oxide nanoparticles according to the present invention.

FIG. 3 is an X-ray diffraction (XRD) pattern of the nanoparticles according to Examples 2 and 3.

FIG. 4 is a transmission electron microscope (TEM) image of the prepared nanoparticles according to Example 2.

FIG. 5 is a transmission electron microscope (TEM) image of the surface-modified hydrophilic nanoparticles according to Example 3.

FIG. 6 shows the result of an X-ray Photoelectron Spectroscopy (XPS) showing the covalently bonded Fe—S according to Example 3.

FIG. 7 shows a picture of the hydrophobic nanoparticles according to Example 2 dispersed in toluene layer (upper layer) and the hydrophilic nanoparticles according to Example 3 dispersed in water layer (lower layer).

FIG. 8 is a transmission electron microscope (TEM) image of the prepared nanoparticles according to Example 4.

FIG. 9 is an infrared spectrum of the prepared nanoparticles according to Example 4.

FIG. 10 is a transmission electron microscope (TEM) image of the prepared nanoparticles according to Example 5.

FIG. 11 is an infrared spectrum of the prepared nanoparticles according to Example 5.

### DETAILED DESCRIPTION OF THE PREFERRED EMBODIMENTS

According to preferable examples of the present invention, uniform-sized hydrophilic iron oxide nanoparticles are produced when superparamagnetic iron oxide particles that are less than 20 nanometers in size and spherical shaped are prepared in an organic solution, thereby achieving the uniformity of the size of the particles, followed by a chemical surface modification, which permits the particles gain their hydrophilic property.

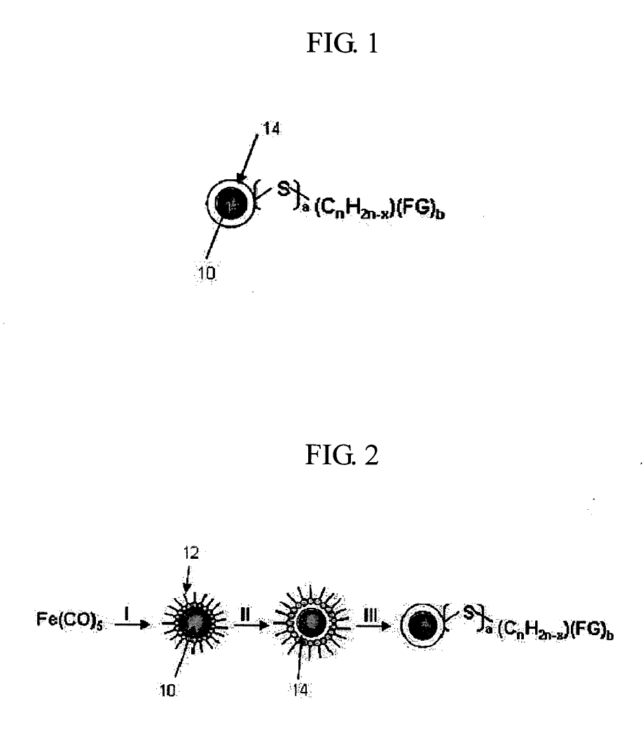

The structure of a metal oxide nanoparticle can be explained with reference to FIG. 1. The surface of a metal oxide nanoparticle 10, also referred to as a core, is coated by a non-stoichiometric metal-rich shell 14, and the shell is strongly bonded by a covalent bond with an element (e.g. sulfur (S) from the examples) of the organic compound. The metal component of the core and the shell is identical. According to FIG. 1, the expression “n” of the organic compound is an integer from 1 to 20; “(CₙH₂ₙ₋ₓ)” indicates hydrocarbon in the form of a chain, a branch or a ring; and “FG” refers to a hydrophilic functional group such as —COOH, —NH₂, —SH. In addition, each of the organic compounds in the FIG. 1 has one (1) to two (2) mercapto (HS—) group(s) and hydrophilic functional groups (FG) capable of providing 1 to 2 covalent bonding sites, and it further provides 1 to 2 potential reaction sites with other compounds (e.g. 3-mercaptopropionic acid, 2-aminoethanethiol, dimercaptosuccinimid acid) in subsequent steps. Furthermore, the core can be in other form of core/shell. In the FIG. 1, “a” and “b” are 1 or 2 respectively, and “x” is selected depending on “a” and “b” as follows:

a=b=1→x=0;

a=1, b=2 or a=2, b=1→x=1;

a=b=2→x=2.

When iron oxide nanoparticles are prepared by making organometallic precursors of iron undergo thermal decomposition in an organic solution containing surfactants, the surface of an iron oxide nanoparticle can be protected by the surfactants, thereby achieving protection, The resulting structure of the nanoparticle has a weak linkage, such as electrostatic interactions or coordinative interaction between the particulate surface and the polar head of the surfactant that has its nonpolar tail facing the outside. When these surface-protected particles are placed into a polar solvent such as water or alcohol, the particles begin agglomerating to form precipitates as the surfactants are immediately stripped off from their surfaces.

Therefore, a chemical bonding, rather than a physical interaction, between a nanoparticle and an organic ligand containing a hydrophilic functional group must be formed in order to achieve chemical stability and dispersity in an aqueous solution. Under normal conditions, the surface of a metallic oxide nanoparticle has poor reactivity, and thus, the particles do not form a chemical bond with an organic ligand.

To overcome these problems, in accordance with a preferable example of the present invention, a thin Fe outer layer (i.e. shell) is formed on the surface of an iron oxide nanoparticle, which has been protected with the surfactants. That is, a Fe-rich layer is formed on the particulate surface. Subsequently, an organic compound, such as 3-mercaptopropionic acid (MPA)[HS(CH₂)₂COOH], covalently bonds with the Fe-rich layer (i.e. shell) on the particulate surface, thus a chemical stability is achieved through the covalent bond of Fe—S and a hydrophilic property is obtained through carboxylic acid.

Iron oxide nanoparticles according to the present invention do not directly form chemical bonds with 3-mercaptopropionic acids. However, by making the outer layer of iron oxide nanoparticles to contain iron-rich composition, covalent bonds (Fe—S) between the iron element of the nanoparticles and the sulfur element of 3-mercaptopropionic acids can be encouraged. Moreover, a carboxylic acid provides a functional group capable of amidation reactions with an amino group abundantly found in biological molecules.

FIG. 2 shows the steps involved in the surface modification of iron oxide nanoparticles.

In the first step (I), iron oxide precursors, Fe(CO)₅, are added to the organic solvent containing surfactants before the solution is heated to reflux, and iron oxide (Fe²⁺ and Fe³⁺ being mixed) nanoparticles are obtained. While the resultant solution is maintained at 80° C., the air is bubbled through it, thereby allowing oxidation, then it is refluxed again to produce a solution containing γ-iron oxide nanoparticles 10. It can be shown that surfactants 12 are attached to the surface of the iron oxide nanoparticles. The surfactants can be one of the following: RNH₂, RCOOH or a combination of the two (R represents alkyl or alkenyl groups and the hydrocarbons are at least 6 chains long). The organic solution can be one of the following: dibenzylether, diphenylether, dioctylether, and octadecene.

In the second step (II), the resultant solution obtained from the first step (I) is maintained at 100° C. while an inert-nitrogen gas is introduced into the solution before adding the precursors, Fe(CO)₅, and then refluxing, thereby forming a metallic layer (that is, shell 14) on γ-iron oxide nanoparticles or making the surface to have non-stoichiometric Fe-rich layer. This technique is based on the principle that iron nanoparticles are produced when Fe(CO)₅ precursors are heated in an inert atmosphere and surfactants-containing organic solution. That is, instead of producing and developing a new core, making an iron shell using the already-existing mechanisms affixed on the surfaces of the iron oxide nanoparticles or making the nanoparticles to have a core and shell structure with non-stoichiometric Fe-rich layer.

In the third step (III), organic compounds containing covalently bonding element with the metal element (Fe) in the nanoparticles, such as 3-mercaptoproprionic acid, is added to the nanoparticles from the second step (II) first, and then it is refluxed to allow the formation of the covalent bonds between iron (Fe) of the nanoparticles and sulfur (S) of 3-mercaptopropionic acids. Or, an alkaline methanolic solution (containing KOH or NaOH) of 3-mercaptoproprionic acid is added to the nanoparticles from the second step (II) at room temperature and then, it is stirred to allow the formation of the covalent bonds between iron (Fe) of the nanoparticles and sulfur (S) of 3-mercaptopropionic acids. The hydrophilic iron oxide nanoparticles are thus formed, exposing carboxyl (COOH) hydrophilic functional group of 3-mercaptopropionic acid. Hence, the hydrophilic property is achieved, which further increases the dispersity of the nanoparticles. Therefore, the nanoparticles also have an advantage of being capable of participating in additional reactions.

Such surface modification can be applied not only to iron oxides, but also to all other kinds of metal oxides. After preparing metal oxide nanoparticles in an organic solution, non-stoichiometric metal-rich layer on the surfaces of the nanoparticles can be formed by adding and heating the precursors of the metal, thereby causing addition reactions. Then, a covalent bond between the metal-rich shell and an organic compound can be formed and the hydrophilic functional group is exposed from the particulate surface. Subsequently, amidation or esterification processes of the nanoparticles with biocompatible polymers or targeting agents or the like can be made possible.

The following are examples showing in detail the preparation of surface-modified hydrophilic metal oxide nanoparticles according to the present invention.

### EXAMPLE 1

Preparation of Hydrophobic γ-Iron Oxide Nanoparticles

1.93 mL (6.09 mmol) of oleic acid was dissolved in 20 mL of dioctylether under nitrogen atmosphere and was maintained at 100° C. Subsequently, 0.40 mL (3.04 mmol) of Fe(CO)₅ precursors were added to the above solution before heating to reflux for 2 hours. Then, the resultant solution was maintained at 80° C. while bubbling the air through it for 16 hours before it was refluxed for another 2 hours. As a result, hydrophobic γ-iron oxide nanoparticles were produced.

### EXAMPLE 2

Layering the Surface of γ-Iron Oxide Nanoparticles

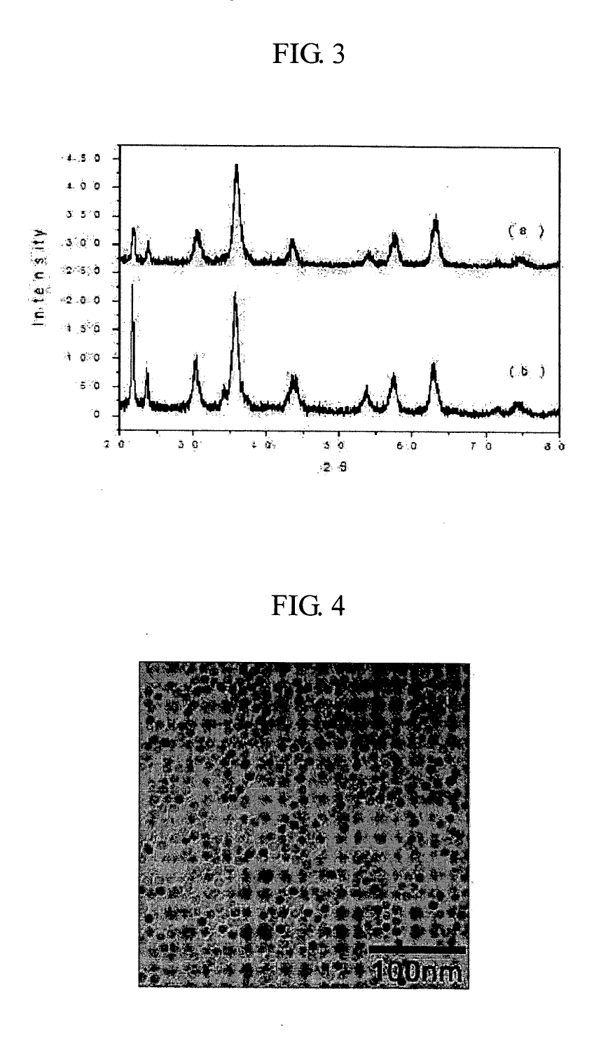

Keeping the resultant solution from Example 1 at 100° C. while bubbling a nitrogen gas through the solution, and then 0.04 mL (0.304 mmol) of Fe(CO)₅ precursors were added subsequently. The solution was, then, refluxed, thereby forming non-stoichiometric iron-rich layer on the surface of the iron oxide nanoparticles. X-ray diffraction patterns and transmission electron microscope images of these nanoparticles are respectively shown in FIG. 3 (a) and 4.

### EXAMPLE 3

Surface modification (I) of γ-Iron Oxide Nanoparticles to Obtain Hydrophilic Property

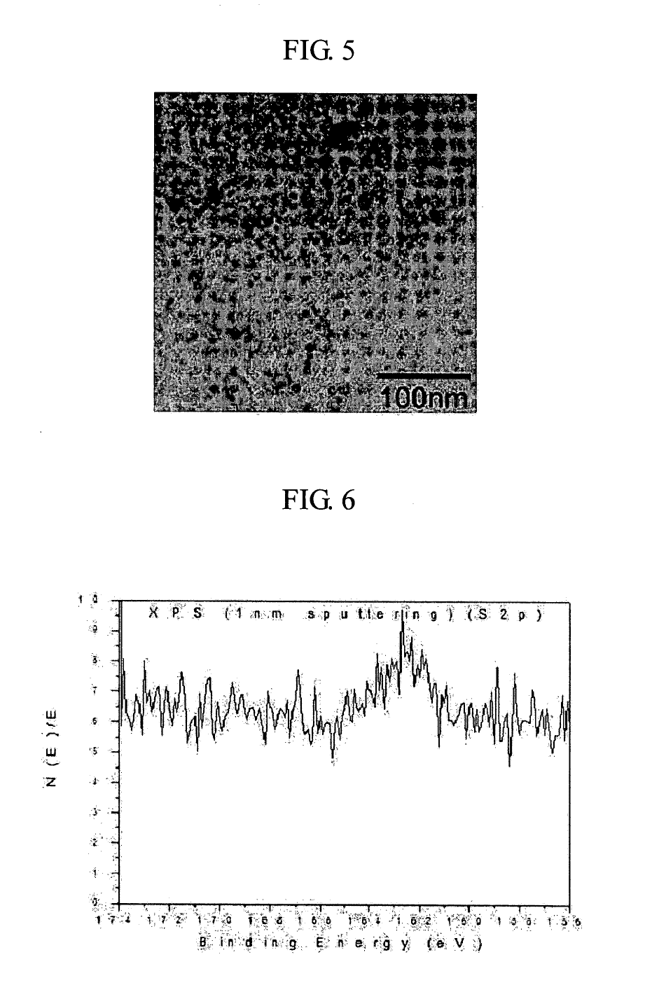

0.039 mL (0.45 mmol) of 3-mercaptopropionic acid was added to the resultant solution from Example 2 before it was refluxed, thereby obtaining chemical stability by the covalent bonding between iron (Fe) and sulfur (S), and furthermore, the carboxyl group is exposed from the surfaces of the nanoparticles, yielding hydrophilic γ-iron oxide nanoparticles. The X-ray diffraction patterns and the transmission electron microscope (TEM) images of these nanoparticles are shown in FIG. 3 (b) and FIG. 5. FIG. 6 illustrates the Fe—S covalent bond characterized from the analysis of the x-ray photoelectron spectroscopy.

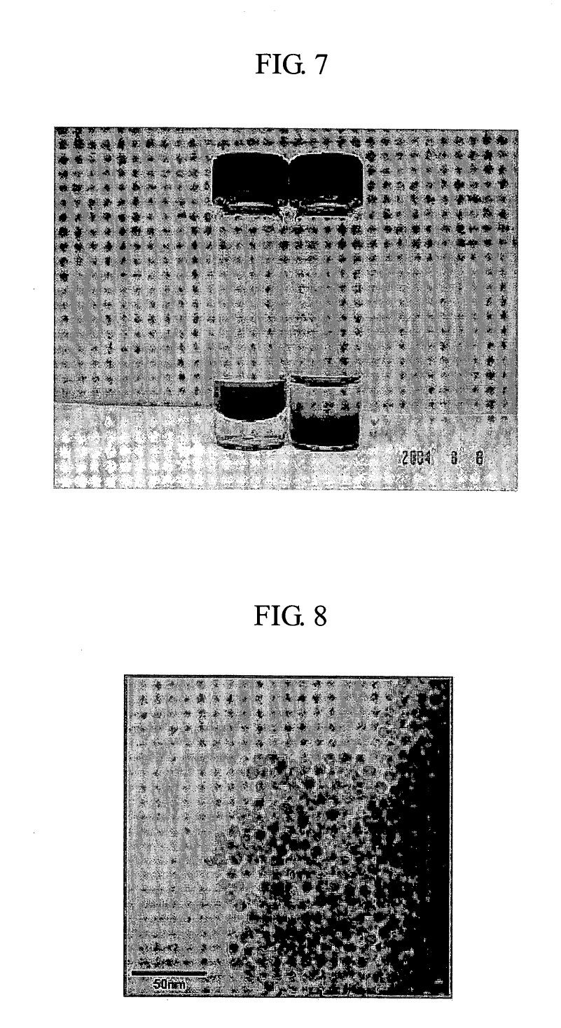

The comparison between Example 2 (before surface-modified) and Example 3 (after surface-modified) that are dispersed in toluene and water is shown on the left and the right test tubes, respectively, in FIG. 7. It can be shown that the surface-modified nanoparticles are well dispersed (as shown on the right).

### EXAMPLE 4

Surface Modification (II) of γ-Iron Oxide Nanoparticles To Obtain Hydrophilic Property

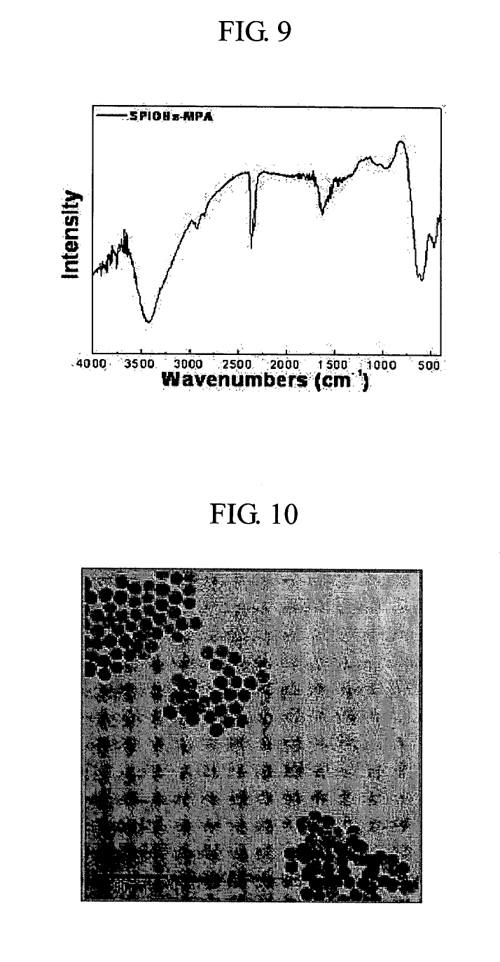

1 mL of the resultant solution from Example 2 was diluted with 25 mL of chloroform (CHCl₃). At room temperature, 0.05 mole/L of 3-mercaptopropionic acid in 3 mL of methanol solution including 0.06 mole/L of NaOH were added to the diluted solution, together with stirring the solution by ultrasonic wave and vortex. Next, 25 mL of water and 25 mL of methanol were added to the solution. Then, nanoparticles were separated using magnet and washed with methanol, thereby obtaining γ-iron oxide nanoparticles with chemical stability by the covalent bonding between iron (Fe) and sulfur (S) and hydrophilic property by the carboxyl group exposed from the surfaces of the nanoparticles. The TEM image and FT-IR spectrum of these nanoparticles are shown in FIGS. 8 and 9. It is found that these nanoparticles have the same dispersity in water and physical and chemical properties as that of the particles of Example 3.

### EXAMPLE 5

Surface Modification (III) of γ-Iron Oxide Nanoparticles To Obtain Hydrophilic Property

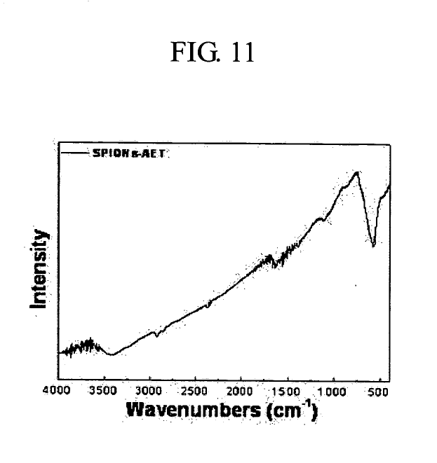

1 mL of the resultant solution from Example 2 was diluted with 25 mL of chloroform (CHCl₃). At room temperature, 0.05 mole/L of 2-aminoethanethiol in 3 ml of methanol solution including 0.11 mole/L of NaOH were added to the diluted solution, together with stirring the solution by ultrasonic wave and vortex. Next, 25 mL of water and 25 mL of methanol were added to the solution. Then, nanoparticles were separated using magnet and washed with methanol, thereby obtaining γ-iron oxide nanoparticles with chemical stability by the covalent bonding between iron (Fe) and sulfur (S) and hydrophilic property by the amine group (NH₂) exposed from the surfaces of the nanoparticles. The TEM image and FT-IR spectrum of these nanoparticles are shown in FIGS. 10 and 11. It is found that these nanoparticles have excellent dispersity in water.

The above explains in detail, by using examples, about metallic oxide-based nanoparticles that can become hydrophilic after they are surface-modified and the method for preparation thereof. However, it should be understood that the above-described embodiments are not limited by any of the details of the foregoing description, unless otherwise specified, but rather should be construed broadly within its spirit and scope as defined in the appended claims, and therefore, all changes and modifications that fall within the metes and bounds of the claims, or equivalence of such metes and bounds are therefore intended to be embraced by the appended claims.

What is claimed is:

1. A metal oxide nanoparticle comprising:
a metal oxide core, coated by a shell consisted of the same metal element as said core; and
an organic compound that contains a hydrophilic functional group and a covalently bonding element that bonds with the metal element of the shell.

2. The nanoparticle of claim 1, wherein said metal element is iron and said organic compound having sulfur as an element that forms a covalent bond with said metal element.

3. The nanoparticle of claim 1, wherein said shell has non-stoichiometric metal-rich composition than said core.

4. A method of preparing a nanoparticle comprising:
preparing metal oxide nanoparticles by making the precursors of the metal oxide undergo thermal decomposition and oxidation processes in an organic solution containing surfactants;
obtaining non-stoichiometric metal-rich layer on the surface of the nanoparticles by adding more precursors to a solution containing the nanoparticles under inert condition and making the mixture undergo thermal decomposition; and
establishing covalent bonds between the metal element of the nanoparticles and organic compounds containing hydrophilic functional groups by adding the organic compounds to the solution containing the nanoparticles.

5. The method of claim 4, wherein the metallic element of the nanoparticle is iron and the organic compound is (HS)ₐ(CₙH₂ₙ₋ₓ) (FG)_{b}, wherein “n” represents an integer selected from 1 to 20, “a” and “b” represents an integer 1 or 2, “FG” represents a hydrophilic functional group and “x” is selected depending on “a” and “b” as follows:
a=b=1→x=0;
a=1, b=2 or a=2, b=1→x=1;
a=b=2→x=2.

6. The method of claim 4, wherein the organic compound is selected from the group consisting of dibenzylether, diphenylether, dioctylether, and octadecene.

7. The method of claim 4, wherein the surfactants are RNH₂ or RCOOH,
wherein R represents an alkyl or an alkenyl, having at least 6 chains of hydrocarbons or a combination of both.

8. The method of claim 4, wherein after adding the organic compounds containing hydrophilic functional groups to the solution containing the nanoparticles, the solution is heated to reflux.

9. A method of preparing a nanoparticle comprising:
preparing metal oxide nanoparticles by making the precursors of the metal oxide undergo thermal decomposition and oxidation processes in an organic solution containing surfactants;
obtaining non-stoichiometric metal-rich layer on the surface of the nanoparticles by adding more precursors to a solution containing the nanoparticles under inert condition and making the mixture undergo thermal decomposition; and
adding an alkaline methanolic solution including organic compounds containing hydrophilic functional groups to the solution containing the nanoparticles at room temperature so as to establish covalent bonds between the metal element of the nanoparticles and the organic compounds.
